(19) United States
(12) Patent Application Publication
(10) Pub. No.: US 2022/0362572 A1
(43) Pub. Date: Nov. 17, 2022

(54) DETACHABLE LIGHT THERAPY BELT

(71) Applicant: SANDER ELECTRONIC CO., LTD., Taipei (TW)

(72) Inventor: JIAJIN HUANG, Taipei (TW)

(73) Assignee: SANDER ELECTRONIC CO., LTD., Taipei (TW)

(21) Appl. No.: 17/319,317

(22) Filed: May 13, 2021

(51) Int. Cl. A61N 5/06; H05K 1/02

(52) U.S. Cl. CPC A61N 5/06; H05K 1/028; H05K 2201/10106; A61N 2005/0645; A61N 2005/0652; A61N 2005/0659; A61N 2005/0664

(57) ABSTRACT

A detachable light therapy belt includes a circuit board, multiple light emitting components, a contact layer, and a mounting component; the circuit board has a first surface and a second surface; the light emitting components are mounted on the first surface of the circuit board; the contact layer has a contact surface, a connecting surface, and multiple holes; the connecting surface and the contact surface face opposite directions, and the connecting surface is connected to the first surface; the holes are formed through the contact layer and on locations corresponding to the light emitting components; the mounting component has a mounting surface, wherein the mounting surface is mounted on the second surface of the circuit board; the contact layer of the detachable light therapy belt contacts a human body, and the connecting surface of the contact layer is detachable from the first surface of the circuit board.

### BACKGROUND OF THE INVENTION

### 1. Field of the Invention

The present invention relates to a light therapy belt, and more particularly a detachable light therapy belt.

### 2. Description of the Related Art

Nowadays, many office workers have to sit at office for a long time. Prolonged sitting causes poor blood circulations for many body parts, and may impact human health.

Furthermore, currently under abnormal climate on a global scale, in recent years hypothermic weathers happen more often than in previous years. People stay warm and maintain healthy blood circulations by either wearing additional clothes, or by using hand warmers for providing warmth to various body parts. However, simply wearing more clothes still may not provide adequate warmth, and using hand warmers for healthy blood circulation is both ineffective and too costly. Additionally, hand warmers are for one-time-use only, implicating environmental problems after each usage.

A device is therefore needed to improve the aforementioned problems, for providing warmth to the human body and for improving blood circulations.

### SUMMARY OF THE INVENTION

To overcome the aforementioned problems, the present invention provides a detachable light therapy belt for providing warmth to a human body and for increasing blood circulations.

The detachable light therapy belt includes a circuit board, multiple light emitting components, a contact layer, and a mounting component. The circuit board has a first surface and a second surface. The light emitting components are mounted on the first surface of the circuit board. The contact layer includes a contact surface, a connecting surface, and multiple holes. The connecting surface and the contact surface face opposite directions, and the connecting surface connects with the first surface of the circuit board. The holes are formed through the contact surface and on locations corresponding to the light emitting components. The mounting component has a mounting surface, and the second surface of the circuit board is mounted on the mounting surface. The detachable light therapy belt of the present invention contacts the human body with the contact surface of the contact layer, and the connecting surface of the contact layer is detachable from the first surface of the circuit board.

The circuit board of the detachable light therapy belt of the present invention is soft and flexible. The light emitting components of the present invention are infrared light emitting diodes (LED), and are mounted on the circuit board. The contact surface of the present invention is a comfortable breathable elastic cloth.

The present invention is wearable by simply fastening a pair of touch fasteners or an elastic inset, and is comfortable under all weather and temperature conditions. The present invention can be fastened on different parts of the human body. Furthermore, the contact surface contacting the human body can be easily detached and washed upon dirtying, rendering the present invention reusable. The detachable light therapy belt of the present invention can be charged multiple times with a direct current (DC) 5V/2 A power supply through a hidden Type C Universal Serial Bus (USB) port, making the present invention more convenient, and making a design of the present invention slimmer.

### BRIEF DESCRIPTION OF THE DRAWINGS

FIG. 1A is an explosion view of a detachable light therapy belt of the present invention.

FIG. 1B is a side view of the detachable light therapy belt of the present invention.

FIG. 2A is a perspective view of an embodiment of the detachable light therapy belt of the present invention.

FIG. 2B is an explosion view of the embodiment of the detachable light therapy belt of the present invention.

FIG. 2C is a cross-sectional view of FIGS. 2A and 2B of the detachable light therapy belt of the present invention.

FIG. 3A is a perspective view of the detachable light therapy belt with a power button.

FIG. 3B is a perspective view of the detachable light therapy belt with a power connection port.

FIG. 4 is a perspective view showing how to use the detachable light therapy belt.

FIG. 5A is a perspective view of the embodiment of the detachable light therapy belt of the present invention with a power module and a control circuit.

FIG. 5B is a perspective view showing how to use the embodiment of the detachable light therapy belt from FIG. 5A.

### DETAILED DESCRIPTION OF THE INVENTION

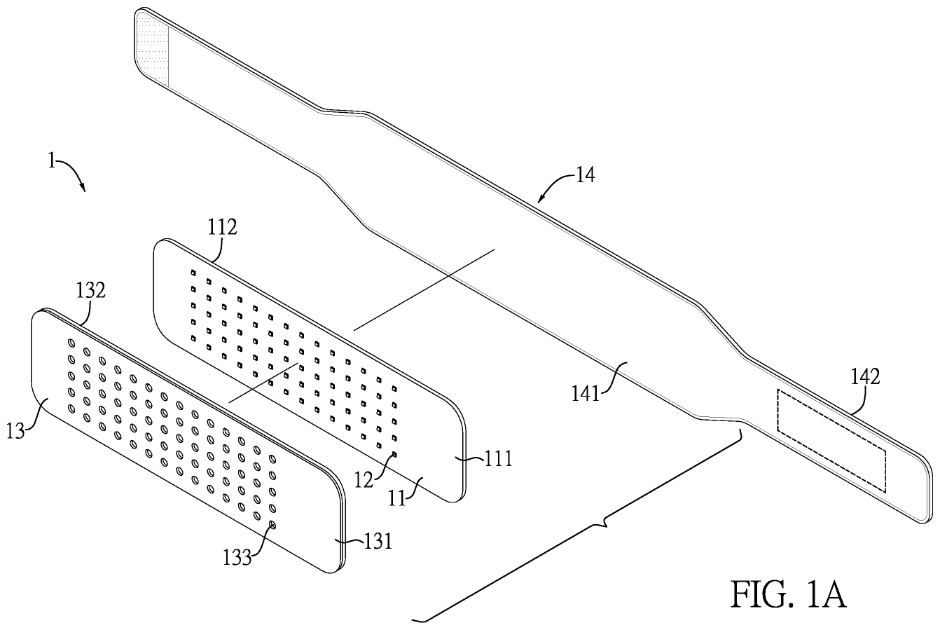

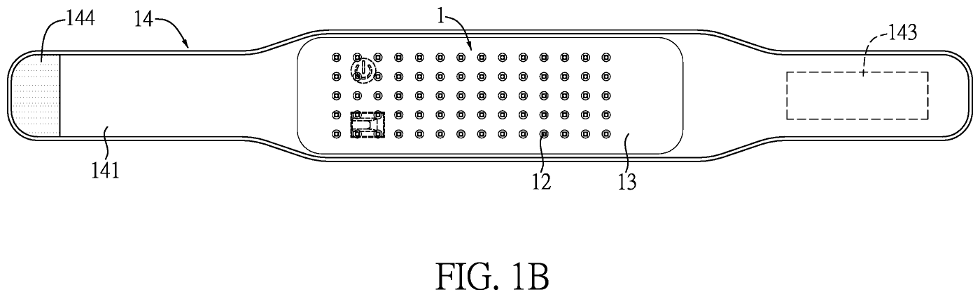

With reference to FIGS. 1A and 1B, an explosion view and a side view of a detachable light therapy belt 1 of the present invention are shown. The detachable light therapy belt 1 includes a circuit board 11, multiple light emitting components 12, a contact layer 13, and a mounting component 14. The circuit board 11 has a first surface 111 and a second surface 112. The light emitting components 12 are mounted on the first surface 111 of the circuit board 11. The contact layer 13 includes a contact surface 131, a connecting surface 132, and multiple holes 133. The connecting surface 132 and the contact surface 131 face opposite directions, and the connecting surface 132 connects with the first surface 111 of the circuit board 11. The holes 133 are formed through the contact surface 13 and on locations corresponding to the light emitting components 12 on the circuit board 11, allowing rays from the light emitting components 12 to shoot out from the holes 133. The mounting component 14 has a mounting surface 141. The second surface 112 of the circuit board 11 is mounted on the mounting surface 141, and the first surface 111 and the second surface 112 of the circuit board 11 face opposite directions. The detachable light therapy belt 1 of the present invention contacts a human body with the contact surface 131 of the contact layer 13, and the connecting surface 132 of the contact layer 13 is detachable from the first surface 111 of the circuit board 11.

In an embodiment of the present invention, the circuit board 11 is a flexible circuit board, enabling the detachable light therapy belt 1 to be flexibly attached to the human body.

In the embodiment of the present invention, the light emitting components 12 are infrared light emitting diodes (LED), and the infrared LEDs emit light with infrared wavelength. The infrared wavelength ranges from 650 to 670 nanometers (nm), or ranges from 840 to 860 nanometers (nm) for the light emitting components 12. The light emitting components 12 can either have a type of infrared LED emitting both wavelengths ranging from 650 to 670 nm and from 840 to 860 nm simultaneously, or have two types of infrared LEDs respectively emitting wavelengths ranging from 650 to 670 nm and from 840 to 860 nm independently. The present invention is open for different light emitting options for the light emitting components 12. In the embodiment of the present invention, the infrared LEDs comprise multiple first infrared LEDs and multiple second infrared LEDs, wherein the first infrared LEDs emit light with infrared wavelengths ranging from 650 to 670 nanometers (nm) and the second infrared LEDs emit light with infrared wavelengths ranging from 840 to 860 nm.

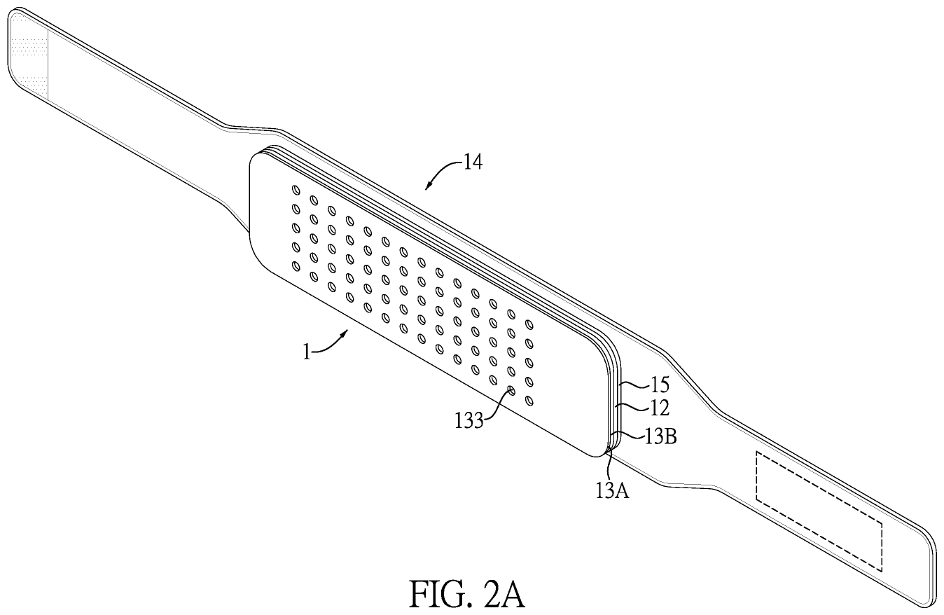

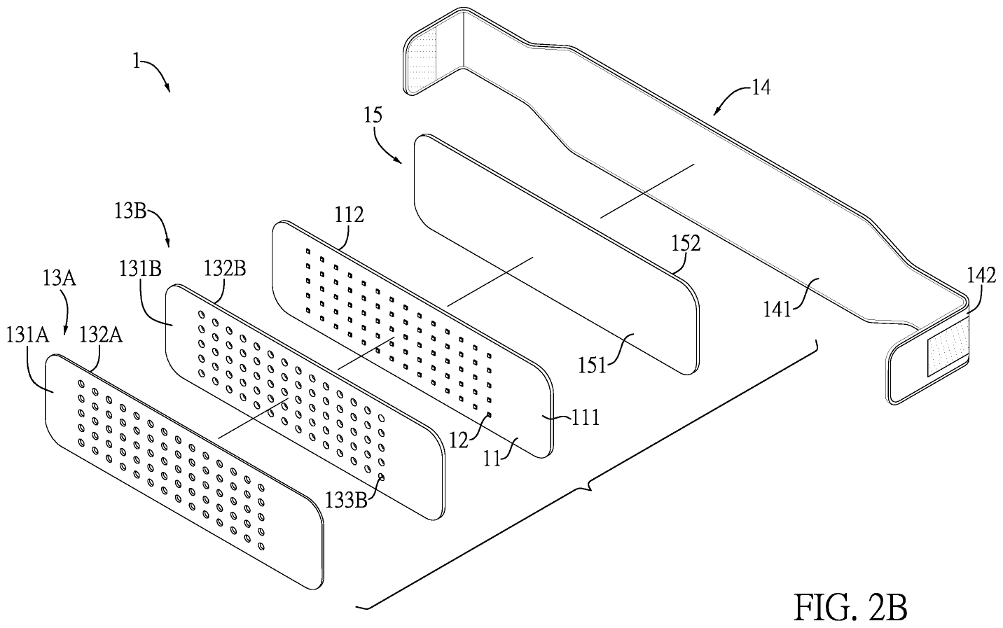

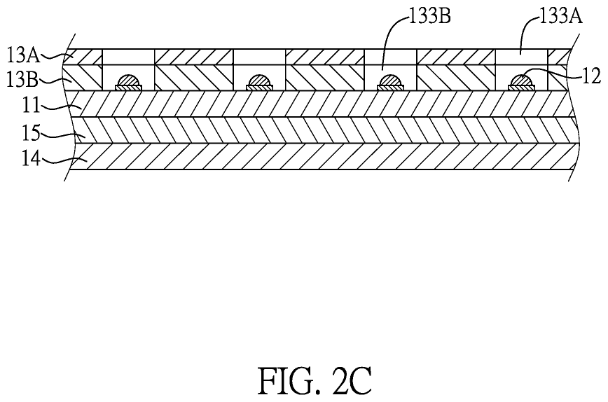

With reference to FIGS. 2A and 2B, a perspective view and an explosion view of another embodiment of the detachable light therapy belt 1 are shown. In this embodiment, the contact layer 13 includes a replaceable cleaning cloth 13A, and a first adhesive cloth 13B. The replaceable cleaning cloth 13A has a front side 131A and a back side 132A. The back side 132A and the front side 131A face opposite directions, and the front side 131A is the contact surface 131. The first adhesive cloth 13B has a first adhesive side 131B and a second adhesive side 132B. The first adhesive side 131B and the second adhesive side 132B face opposite directions. The back side 132A of the replaceable cleaning cloth 13A is detachably adhered to the first adhesive side 131B of the first adhesive cloth 13B, and the second adhesive side 132B is the connecting surface 132. In this embodiment of the present invention, the first adhesive side 131B of the first adhesive cloth 13B is made of a flexible fabric, and the second adhesive side 132B of the first adhesive cloth 13B is adhered to the first surface 111 of the circuit board 11. As the first adhesive side 131B is detachably adhered to the back side 132A of the replaceable cleaning cloth 13A, the first adhesive cloth 13B can be detached and cleaned when cleaning the replaceable cleaning cloth 13A.

With reference to FIGS. 2B and 2C, FIG. 2C is a cross-sectional view of FIGS. 2A and 2B of the detachable light therapy belt 1 of the present invention. The replaceable cleaning cloth 13A and the first adhesive cloth 13B respectively have the holes 133A, 133B. Each hole 133A, 133B is formed through the replaceable cleaning cloth 13A and the first adhesive cloth 13B, and each hole 133A, 133B is formed correspondingly to locations of the light emitting components 12 on the circuit board 11. A depth of any one of the holes 133 is greater than a height of any one of the light emitting components 12, preventing the light emitting components 12 from direct-contacting and overheating the human body parts when the detachable light therapy belt 1 is being used.

With reference to FIGS. 2A, 2B and 2C, the detachable light therapy belt 1 further includes a second adhesive cloth 15. Mounted between the mounting component 14 and the circuit board 11, the second adhesive cloth 15 has a first adhesive side 151 and a second adhesive side 152. The first adhesive side 151 and the second adhesive side 152 face opposite directions. The second surface 112 of the circuit board 11 is mounted on the first adhesive side 151 of the second adhesive cloth 15, and the second adhesive side 152 of the second adhesive cloth 15 is mounted on the mounting surface 141 of the mounting component 14. Furthermore, in this embodiment of the present invention, the circuit board 11, the first adhesive cloth 13B, and the second adhesive cloth 15 can be connected by sewing, protecting the light emitting components 12 and other electronic components on the circuit board 11, and further preventing the first adhesive cloth 13B and the second adhesive cloth 15 from falling off from the first surface 111 and the second surface 112 of the circuit board 11.

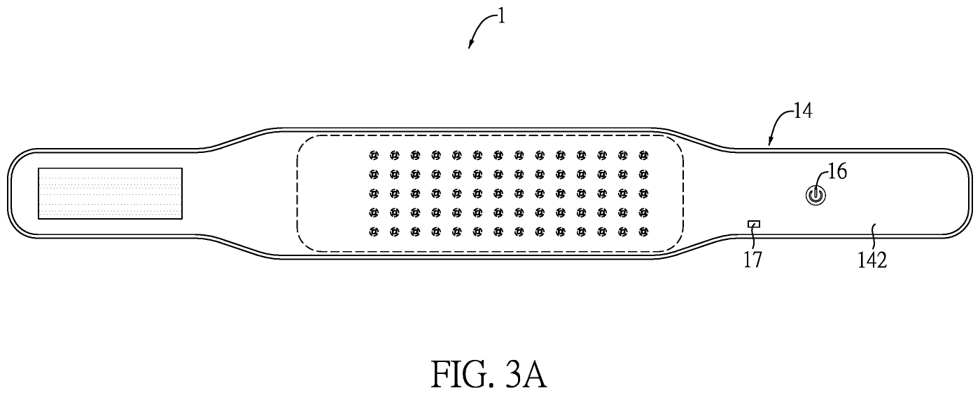

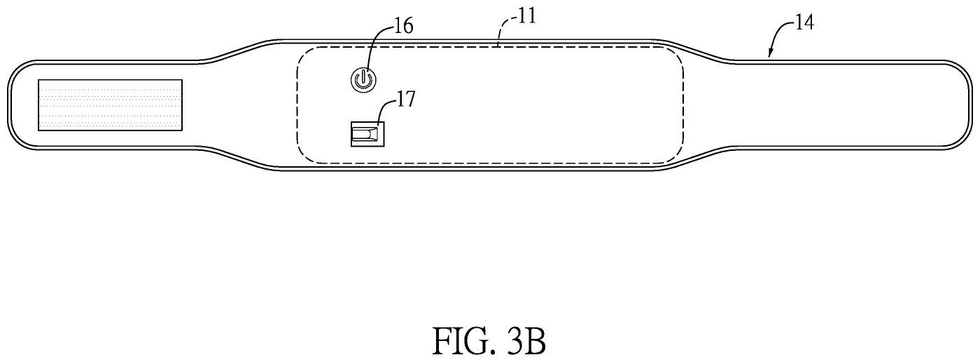

With reference to FIGS. 3A and 3B, perspective views of the detachable light therapy belt 1 with a power button 16 and a power connection port 17 are shown. The detachable light therapy belt 1 includes a power button 16 and a power connection port 17. The power button 16 is electrically connected with the circuit board 11 and the power connection port 17 for switching the light emitting components 12 on and off. In the embodiment of the present invention, the power button 16 is either mounted on a surface 142 of the mounting component 14. The surface 142 and the mounting surface 141 face opposite directions, or the power button 16 is mounted on the circuit board 11, for ease of accessing and pushing the power button 16 when the detachable light therapy belt 1 is in use on the human body.

The power connection port 17 is electrically connected to an external power source, and the power connection port 17 can be either mounted on the surface 142 of the mounting component 14 or on the circuit board 11. In the embodiment of the present invention, the power connection port 17 is a Type-C Universal Serial Bus (USB), and the power connection port 17 is connectable to receive electricity from a portable power source, providing electricity for the light emitting components 12 on the circuit board 11.

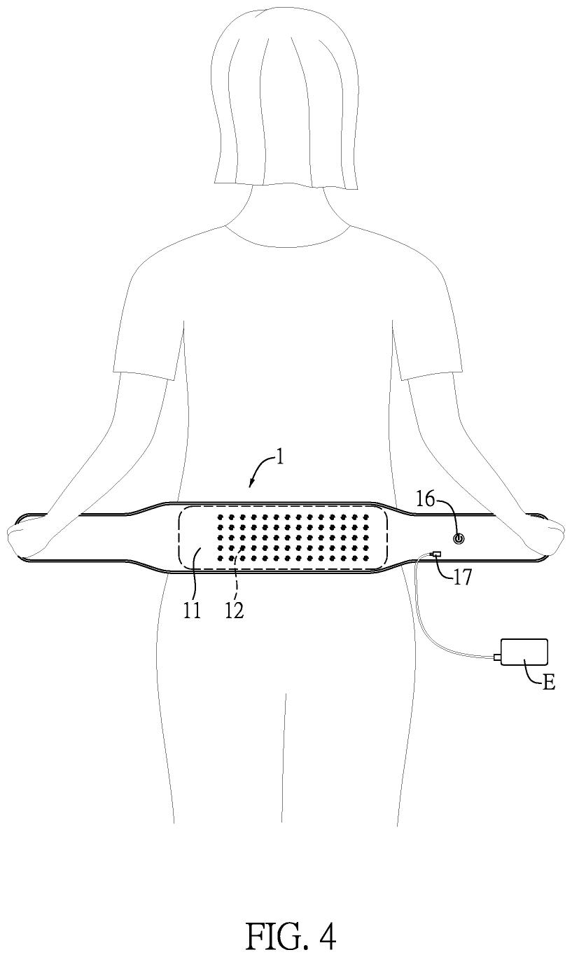

With reference to FIGS. 1B and 4, FIG. 4 is a perspective view showing how to use the detachable light therapy belt 1 of the present invention. In FIG. 1B, the mounting component 14 includes a pair of touch fasteners 144 and a bonding part 143. The touch fasteners 144 are mounted on the mounting surface 141 and are located on a side of the mounting component 14. The bonding part 143 is mounted on the surface 142 of the mounting component 14 and is located on another side of the mounting component 14. In FIG. 4, when the detachable light therapy belt 1 is in use, a user can adjust relative positions of the touch fasteners 144 and the bonding part 143 according to how the human body is receiving the treatment. The detachable light therapy belt 1 can be accordingly fastened around joints, waist, back, or neck and shoulders of the human body with the mounting component 14 while the power connection port 17 is connected to an external power source E. In addition, in another embodiment of the present invention, an elastic inset can be used in replacement of the touch fasteners 144 for adjusting the mounting component 14.

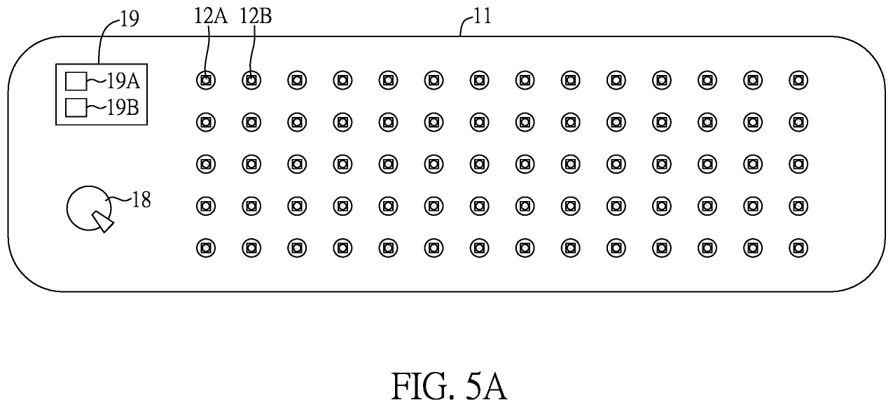

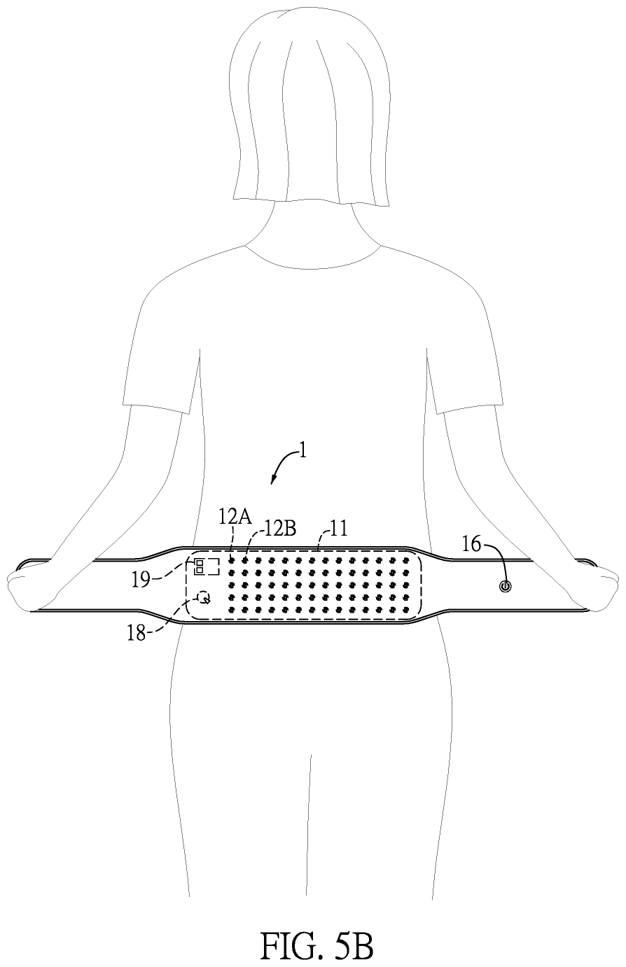

With reference to FIGS. 5A, a perspective view of another embodiment of the detachable light therapy belt 1 of the present invention with a power module 18 and a control circuit 19. FIG. 5B is perspective view showing how to use the embodiment of the detachable light therapy belt 1 from FIG. 5A. In this embodiment, the detachable light therapy belt 1 includes multiple light emitting components 12A, multiple light emitting components 12B, the power button 16, a power module 18, and a control circuit 19. The power button 16 is electrically connected with the circuit board 11 and the power module 18 for switching the light emitting components 12A and the light emitting components 12B on and off. The light emitting components 12A and the light emitting components 12B respectively emit infrared wavelengths ranging from 650 to 670 nm and from 840 to 860 nm. Wavelengths from 840 to 860 nm are outside of the visual spectrum. The light emitting components 12A and the light emitting components 12B are alternatingly mounted on the circuit board 11. Furthermore, different from other embodiments of the present invention, the power module 18 in this embodiment of the present invention is able to provide electricity to the light emitting components 12A and the light emitting components 12B without an external power source.

The light emitting components 12A and the light emitting components 12B in the embodiment mentioned earlier are respectively the first infrared LEDs and the second infrared LEDs, wherein the first infrared LEDs and the second infrared LEDs are alternatingly mounted on the circuit board 11.

In addition, the control circuit 19 further includes a memory module 19A and a data transmission module 19B. The memory module 19A and the data transmission module 19B both are electrically connected to the light emitting components 12A, the light emitting components 12B, and the power module 18. The memory module 19A is used to record a light emitting time of the light emitting components 12A and the light emitting components 12B. The data transmission module 19B is electrically connected to the memory module 19A, and the data transmission module 19B transmits the light emitting time of the light emitting components 12A and the light emitting components 12B to either a cloud network, a smart device, or a personal computer, for displaying a status of the detachable light therapy belt 1.

Similarly, in the perspective view of FIG. 5B, when using the detachable light therapy belt 1, the user can adjust the relative positions of the touch fasteners 144 and the bonding part 143 according to how the human body is receiving the treatment. The detachable light therapy belt 1 can be accordingly fastened around the joints, the waist, the back, or the neck and shoulders of the human body with the mounting component 14, or alternatively using the elastic inset in replacement of the touch fasteners 144 for adjusting the mounting component 14.

In another embodiment of the present invention, the power module 18 and the power connection port 17 can be both included in the detachable light therapy belt 1. In this case, the light emitting components 12, the light emitting components 12A, and the light emitting components 12C on the circuit board 11 can be either powered by the external power source or independently powered by the power module 18 according to a preference of the user.

In conclusion, the circuit board 11 of the detachable light therapy belt 1 of the present invention can be soft and flexible. The infrared LEDs can be mounted on the circuit board 11. Breathable elastic cloth can be adhesively combined with the circuit board 11. Using the present invention simply requires fastening the touch fasteners 144 or the elastic inset, and is comfortable under all weather and temperature conditions. The present invention can be fastened on different parts of the human body. Furthermore, the contact surface 13 contacting the human body can be easily detached and washed upon dirtying, rendering the present invention reusable. Instead of mounting a bulky battery, the detachable light therapy belt 1 of the present invention can be charged multiple times with a direct current (DC) 5V/2 A power supply through a hidden Type C Universal Serial Bus (USB), making the present invention more convenient, and making a design of the present invention slimmer.

What is claimed is:

1. A detachable light therapy belt, comprising:
a circuit board, having a first surface and a second surface, wherein the first surface and the second surface face opposite directions;
multiple light emitting components, mounted on the first surface of the circuit board;
a contact layer, comprising:
a contact surface;
a connecting surface, connecting with the first surface of the circuit board, wherein the connecting surface and the contact surface face opposite directions; and
multiple holes, formed through the contact surface and on locations corresponding to the light emitting components; and
a mounting component, having a mounting surface, wherein the mounting surface is mounted on the second surface of the circuit board;
wherein the contact surface of the contact layer is configured to contact a human body, and the connecting surface of the contact layer is detachable from the first surface of the circuit board.

2. The detachable light therapy belt as claimed in claim 1, wherein the circuit board is a flexible circuit board.

3. The detachable light therapy belt as claimed in claim 1, wherein the light emitting components are infrared light emitting diodes (LED).

4. The detachable light therapy belt as claimed in claim 1, wherein the contact layer comprises:
a replaceable cleaning cloth, having a front side and a back side, wherein the back side and the front side face opposite directions, and the front side is the contact surface; and
a first adhesive cloth, having a first adhesive side and a second adhesive side, wherein the first adhesive side and the second adhesive side face opposite directions, and the first adhesive side is detachably mounted on the back side of the replaceable cleaning cloth; the second adhesive side is the connecting surface.

5. The detachable light therapy belt as claimed in claim 4, further comprising:
a second adhesive cloth, mounted between the mounting component and the circuit board; wherein the second adhesive cloth has a first adhesive side and a second adhesive side facing opposite directions, the second surface of the circuit board is mounted on the first adhesive side of the second adhesive cloth, and the second adhesive side of the second adhesive cloth is mounted on the mounting surface of the mounting component.

6. The detachable light therapy belt as claimed in claim 5, wherein the circuit board, the first adhesive cloth, and the second adhesive cloth are connected by sewing.

7. The detachable light therapy belt as claimed in claim 1, wherein the mounting component comprises a pair of touch fasteners and a bonding part, the touch fasteners are mounted on the mounting surface of the mounting component, the bonding part is mounted on a surface of the mounting component, and the mounting surface and the surface face opposite directions.

8. The detachable light therapy belt as claimed in claim 1, wherein the contact surface of the contact layer is configured to contact joints, waist, back, or neck and shoulders of the human body.

9. The detachable light therapy belt as claimed in claim 1, further comprising:
a power button, electrically connecting the circuit board for switching the light emitting components on and off.

10. The detachable light therapy belt as claimed in claim 1, further comprising:
a power connection port, electrically connecting an external power source.

11. The detachable light therapy belt as claimed in claim 10, wherein the power connection port is a Type C Universal Serial Bus (USB).

12. The detachable light therapy belt as claimed in claim 3, wherein the infrared LEDs emit light with infrared wavelengths ranging from 650 to 670 nanometers (nm).

13. The detachable light therapy belt as claimed in claim 3, wherein the infrared LEDs emit light with infrared wavelengths ranging from 840 to 860 nanometers (nm).

14. The detachable light therapy belt as claimed in claim 3, wherein the infrared LEDs comprise multiple first infrared LEDs and multiple second infrared LEDs;
wherein the first infrared LEDs emit light with infrared wavelengths ranging from 650 to 670 nanometers (nm) and the second infrared LEDs emit light with infrared wavelengths ranging from 840 to 860 nm.

15. The detachable light therapy belt as claimed in claim 14, wherein the first infrared LEDs and the second infrared LEDs are alternatingly mounted on the circuit board.
